(19) United States
(12) Patent Application Publication
(10) Pub. No.: US 2006/0093634 A1
(43) Pub. Date: May 4, 2006

(54) Personal care compositions and concentrates for making the same

(75) Inventors: Patrick J. Lutz, Nazareth, PA (US); Kristy A. Winters, Middlesex, NJ (US); Kelly A. Dunn, High Bridge, NJ (US); Jacob Guth, Upper Black Eddy, PA (US)

Correspondence Address: DARBY & DARBY P.C., P. O. BOX 5257, NEW YORK, NY, 10150-5257, US

(73) Assignee: Lonza Inc., Fair Lawn, NJ (US)

(21) Appl. No.: 11/112,479

(22) Filed: Apr. 22, 2005

Related U.S. Application Data
(60) Provisional application No. 60/565,003, filed on Apr. 23, 2004.

(51) Int. Cl. A61K 8/37; A61K 8/49

(52) U.S. Cl. 424/401

(57) ABSTRACT

The present invention relates to color stable personal care compositions that provide anti-oxidiation, moisturization, and/or prevent biodegradation in personal care formulations. These compositions contain erythorbic acid or a salt thereof, caffeic acid or a salt thereof, δ-gluconolactone, or a mixture thereof. The present invention further relates to methods of moisturizing skin with erythorbic acid or a salt thereof, or δ-gluconolactone. The present invention also relates to concentrates of active ingredients preloaded with a sufficient amount of an anti-oxidation, moisturizing and/or biodegradation prevention agent so that when it is diluted for use, the final formulation includes an anti-oxidation, moisturizing and/or biodegradation prevention effective amount of the anti-oxidation, moisturizing, and/or biodegradation prevention agent.

This application claims the benefit of U.S. Provisional Application No. 60/565,003, filed Apr. 23, 2004, which is hereby incorporated by reference.

### FIELD OF THE INVENTION

The present invention relates to color stable personal care compositions that provide anti-oxidation and/or moisturization, and/or prevent biodegradation in personal care formulations. These compositions contain erythorbic acid or a salt thereof, caffeic acid or a salt thereof, α-gluconolactone, or a mixture thereof. The present invention further relates to methods of moisturizing skin with erythorbic acid or a salt thereof, or δ-gluconolactone. The present invention also relates to concentrates of active ingredients preloaded with a sufficient amount of an anti-oxidation, moisturizing, and/or biodegradation prevention agent so that when it is diluted for use, the final formulation includes an anti-oxidation, moisturizing, and/or biodegradation prevention effective amount of the anti-oxidation, moisturizing, and/or biodegradation prevention agent.

### BACKGROUND OF THE INVENTION

Natural products, while often safe, generally have color and odor stability problems in blends and finished formulations due to chemical interaction or biodegradation. Many commonly used natural products darken or develop odors when kept at room temperature.

There is a continuing need for low cost and safe personal care systems which are color stable and have no odor.

### SUMMARY OF THE INVENTION

Erythorbic acid or salts thereof, caffeic acid or salts thereof, and δ-gluconolactone frequently cause color change in formulations. Surprisingly, it has been found that formulations containing sodium erythorbate or caffeic acid can be stabilized by incorporating sodium sulfite into the formulation. It has also been surprisingly found that sodium erythorbate and δ-gluconolactone moisturize skin more effectively than many known moisturizers, such as glycerin.

One embodiment of the present invention is a color stable personal care composition comprising:
(a) an anti-oxidation, moisturizing, and/or biodegradation preventing (AMBP) component comprising:
   (1) erythorbic acid or a salt thereof;
   (2) caffeic acid or a salt thereof;
   (3) δ-gluconolactone; or
   (4) a mixture of at least two of the foregoing; and
(b) a color stabilizing effective amount of a color stabilizer selected from a sulfite, a sulfate, a thioglycolate, a gluconate, or a mixture thereof.

Preferably, the color stable personal care composition includes an anti-oxidation, moisturization, and/or biodegradation preventive effective amount (such as a microorganism inhibiting, bactericidal, and/or fungicidal amount) of the AMBP component. The color stable composition may be, for example, for topical application to the skin, hair, and/or scalp. The color stabilizer of the present invention can also improve biodegradation prevention efficacy of the AMBP component. According to one preferred embodiment, the color stable composition includes an anti-oxidation and/or biodegradation prevention potentiating effective amount of the color stabilizer.

The color stable personal care composition may include one or more additional anti-microbial agents (e.g., preservatives). Other suitable antimicrobial agents include, but are not limited to, sorbic acid, benzoic acid, and salts thereof, and mixtures thereof.

Preferably, the AMBP component comprises sodium erythorbate.

More preferably, the AMBP component further comprises benzoic acid or a salt thereof, preferably sodium benzoate, or sorbic acid or a salt thereof, preferably potassium sorbate

Particularly preferred AMBP components and mixtures include, but are not limited to, those shown in the table below.

| Mixture | | |
|---|---|---|
| No. | Component 1 | Component 2 |
| 1 | Erythorbic acid or a salt thereof, | δ-Gluconolactone |
| | preferably sodium erythorbate | |
| 2 | Erythorbic acid or a salt thereof, | — |
| | preferably sodium erythorbate | |
| 3 | Erythorbic acid or a salt thereof, | Benzoic acid or a salt thereof, |
| | preferably sodium erythorbate | preferably sodium benzoate |
| 4 | Erythorbic acid or a salt thereof, | Sorbic acid or a salt thereof, |
| | preferably sodium erythorbate | preferably potassium sorbate |
| 5 | Erythorbic acid or a salt thereof, | Caffeic acid or a salt thereof |
| | preferably sodium erythorbate | |
| 6 | Caffeic acid or a salt thereof | — |
| 7 | Caffeic acid or a salt thereof | Benzoic acid or a salt thereof, |
| | | preferably sodium benzoate |
| 8 | Caffeic acid or a salt thereof | Sorbic acid or a salt thereof, |
| | | preferably potassium sorbate |
| 9 | δ-Gluconolactone | — |
| 10 | δ-Gluconolactone | Benzoic acid or a salt thereof, |
| | | preferably sodium benzoate |
| 11 | δ-Gluconolactone | Sorbic acid or a salt thereof, |
| | | preferably potassium sorbate |
| 12 | δ-Gluconolactone | Caffeic acid or a salt thereof |

Another embodiment is a personal care composition comprising:
(a) an anti-oxidation, moisturizing, and/or biodegradation preventive effective amount of a AMBP component; and
(b) an anti-oxidation and/or biodegradation prevention potentiator selected from a sulfite, a sulfate, a thioglycolate, a gluconate, or mixture thereof.

Another embodiment is a method of killing and/or inhibiting the growth of microorganisms on a substrate or in a product comprising applying or including an effective amount of any of the personal care compositions of the present invention, which include a biodegradation prevention effective amount of biodegradation prevention agents (such as any of the AMBP components and/or antimicrobial agents), to the substrate or in the product.

Yet another embodiment is a method of preserving a product (e.g., a personal care product) while maintaining its color comprising incorporating a biodegradation preventive effective amount of the color stable personal care composition of the present invention into the product.

Yet another embodiment is a method of stabilizing the color of a composition containing one or more of erythorbic acid and salts thereof, caffeic acid and salts thereof, and δ-gluconolactone comprising adding or including a color stabilizing effective amount of a sulfite or sulfate to the composition.

Yet another embodiment is a product comprising an antimicrobial, preservative, bactericidal, and/or fungicidal effective amount of any of the personal care compositions of the present invention, such as the color stable personal care composition.

The present invention also relates to a non-therapeutic method of moisturizing skin by applying a moisturizing effective amount of a moisturizing agent selected from erythorbic acid or a salt thereof (e.g., sodium erythorbate), δ-gluconolactone, or a mixture thereof. According to one embodiment, a moisturizing effective amount of the color stable personal care composition of the present invention is applied.

Another embodiment is a moisturizing composition for topical application (e.g., to skin, hair, or scalp) comprising a moisturizing effective amount, preferably from 0.01% to 1% by weight, of sodium erythorbate, δ-gluconolactone, or a mixture thereof.

The present invention also relates to concentrates of active ingredients (e.g., hair and skin active ingredients) preloaded with a sufficient amount of a biodegradation prevention agent so that when it is diluted for use, the final formulation includes a biodegradation prevention effective amount of the biodegradation prevention agent.

One embodiment is a preloaded concentrate comprising:
(a) at least about 1, 2, or 5% by weight of active ingredients (e.g., hair and skin active ingredients) other than a biodegradation prevention agent; and
(b) at least about 1, 2, or 5% by weight of a biodegradation prevention agent.

According to one embodiment, the preloaded concentrate only contains 8, 7, 6, 5, 4, 3, or 2 ingredients.

Generally, the preloaded concentrate is physically and chemically stable. Preferably, the preloaded concentrate is color stable. The concentrate may also include one or more color stabilizers. Suitable color stabilizers include, but are not limited to, those discussed above, such as sodium sulfite, as well as those described in U.S. Pat. No. 6,017,955. The concentration of color stabilizer generally ranges from about 1 to about 40% by weight, based on 100% total weight of concentrate.

Yet another embodiment is a method of preparing a personal care product comprising the step of mixing:
(a) a preloaded concentrate comprising:
   (i) at least about 5% by weight of one or more active ingredients (e.g., hair and skin active ingredients) other than a biodegradation prevention agent, and
   (ii) at least about 1 or 2% by weight of a biodegradation prevention agent; with
(b) one or more additives.

The resulting personal care product comprises a biodegradation prevention effective amount of the biodegradation prevention agent. The preloaded concentrate may include a color stabilizer as discussed above. According to one embodiment, at least one of the additives is not a solvent.

According to one preferred embodiment, the preloaded concentrate comprises
(i) (a) at least about 1% by weight of arbutin, and
   (b) (1) at least about 1% by weight of glycolic acid or a salt thereof,
      (2) at least about 1% by weight of salicylic acid or a salt thereof,
      (3) at least about 1% by weight of L-carnitine, an acyl L-carnitine, or a mixture thereof,
   (4) at least about 1% by weight of vegetable oil microspheres (oleosomes), or
   (5) a mixture thereof; and
(ii) at least about 1 or 2% by weight of a biodegradation prevention agent. Preferred mixtures in component (i) include, but are not limited to, (1) arbutin and glycolic acid, (2) arbutin and salicylic acid, and (3) arbutin and L-carnitine. For example, the preloaded concentrate can include
(i) (a) from about 1, 2, or 5% to about 10, 15, or 20% by weight of arbutin, and
   (b) (1) from about 1, 2, or 5% to about 10, 15, 20, 25, or 28% by weight of glycolic acid or a salt thereof,
   (2) from about 1, 2, or 5% to about 10, 15, or 20% by weight of salicylic acid or a salt thereof,
   (3) at least about 1, 2, 5, or 10% to about 15, 20, 25, 30, 40, or 50% by weight of L-carnitine, an acyl L-carnitine, or a mixture thereof,
   (4) at least about 1% by weight of vegetable oil microspheres (oleosomes), or
   (5) a mixture thereof; and
(ii) at least about 1 or 2% by weight of a biodegradation prevention agent.
   The preloaded concentrate preferably includes a color stabilizer. The concentration of color stabilizer generally ranges from about 1 or 2% to about 10, 20, 30, or 40% by weight, based on 100% total weight of concentrate.

### BRIEF DESCRIPTION OF THE DRAWINGS

FIG. 1 is a bar graph of the skin moisture of (a) untreated skin, (b) skin treated with a 2% glycerin solution (Sample #1), (c) skin treated with a 1% aqueous solution of sodium erythorbate (Sample #2), (d) skin treated with a 1% aqueous solution of 6-gluconolactone (Sample #3), and (e) skin treated with a 2% aqueous solution of a biodegradation prevention mixture (75% (by weight) sodium benzoate and 25% (by weight) sodium erythorbate) (Sample #4).

### DETAILED DESCRIPTION OF THE INVENTION

Definitions

The term “microorganisms” includes, but is not limited to, bacteria, fungi, yeasts and algae.

The term “personal care products” refers to products intended for application (e.g., topical application) to the human body, such as to skin, hair, scalp and nails, including, but not limited to, shampoos, conditioners, creams, lotions (such as body lotions), cosmetics, and soaps.

The term “potentiating” refers to the ability of a compound or composition to enhance or increase the biodegradation prevention efficacy of a biodegradation prevention agent. Preferably, the efficacy of the combined mixture is greater than the additive effect of the ingredients, i.e., the combination is synergistic.

Suitable salts of sorbic acid, erythorbic acid, and benzoic acid include, but are not limited to, pharmaceutically acceptable salts, such as alkali metal or alkali earth metal salts thereof, (e.g., potassium and sodium salts).

Color Stabilizer

The color stable personal care composition preferably contains at least about 0.1% by weight of the color stabilizer or potentiator, based on 100% weight of the personal care composition. More preferably, the color stable personal care composition contains from about 0.01 to about 10%, and even more preferably from about 0.1 to about 1% by weight of the color stabilizer or potentiator. The amount of the color stabilizer needed to stabilize the ingredients in the composition may vary depending on, among other things, the amount of erythorbic acid, caffeic acid, and 6-gluconolactone present and can be determined by one skilled in the art. Similarly, the amount of potentiator to improve the anti-oxidation and/or biodegration efficacy of the AMBP component may vary depending on, among other things, the amount of and type of AMBP component.

Preferred color stabilizers or potentiators include, but are not limited to, sodium sulfite, sodium thioglycolate, calcium gluconate, sodium gluconate, and mixtures of at least two of the foregoing.

Other suitable color stabilizers which may be used in the personal care compositions include those described in U.S. Pat. No. 6,017,955.

AMBP Component

A preferred salt of sorbic acid is potassium sorbate.

A preferred salt of erythorbic acid is sodium erythorbate.

A preferred salt of benzoic acid is sodium benzoate.

Additional antimicrobial agents, e.g., preservatives, may be included in the color stable personal care composition.

Suitable preservatives are known in the art. They include, but are not limited to, quaternary ammonium compounds such as quaternary ammonium carbonates and quaternary ammonium chlorides like benzalkonium chloride; iodine containing compounds, such as 3-iodo-2-propynyl butyl carbamate (IPBC); hydantoins, such as dimethylhydantoin and halogenated hydantoins; isothiazolinones; parabens, such as methylparaben, ethylparaben, and propylparaben; dehydroacetic acid and salts thereof; isocil; chloroxylenol; chlorhexidine; antimicrobial alcohols, such as phenoxyethanol, benzyl alcohol, phenethyl alcohol and chlorobutanol; salicylic acid and salts thereof; triclosan; triclocarban; chlorophenesin; polyaminopropyl biguanide; natural oils (e.g., tea tree oils); and any combination of any of the foregoing.

Preferred antimicrobial compositions for inclusion in the personal care composition include, but are not limited to, those described in International Publication No. WO 2004/014416.

Generally, the active component of the antimicrobial composition is present in the personal care composition at from about 0.1 to about 2.0% by weight, based on 100% total weight of color stable personal care composition.

According to one embodiment, the antimicrobial composition contains at least 0.1% by weight of sorbic acid or a salt thereof (e.g., potassium sorbate), benzoic acid or a salt thereof (e.g., sodium benzoate), or a mixture thereof.

Other Additives

Other additives may be included in the color stable personal care composition as known in the art. Suitable additives include, but are not limited to, solubilizing agents; chelating agents, such as ethylenediaminetetraacetic acid (EDTA) and salts thereof and zeolites; surfactants, such as cationic, anionic, nonionic, and amphoteric surfactants; antioxidants, such as butylated hydroxyanisole (BHA) and butylhydroxytoluene (BHT); amine oxides; tertiary amines; zinc compounds; hydrotropes; fluoride compounds; magnesium salts; calcium salts; carboxylic acids; phosphates; phosphonates; formaldehyde donors; glycereth-7; myristyl myristate; glutaraldehydes; biguanides; reducing agents (such as sulfites); natural products, such as geraniol, usnic acid, and tea tree oils; and any combination of any of the foregoing. Other suitable additives include those listed as active ingredients and additives with regard to the preloaded concentrate below.

The personal care composition may include a solvent.

Suitable solvents are e.g. water and water miscible solvents, including, but not limited to, alcohols (e.g., methanol, ethanol, propanol, isopropyl alcohol, and butanol), glycols (e.g. glycerin, diglycerin, butylene glycol, butoxydiglycol, propylene glycol, 1,2-hexanediol, caprylyl glycol (1,2-octanediol) and dipropylene glycol), glyceryl caprate, glyceryl caprylate, caprylic/capric glycerides, phospholipids, butylene glycol monopropionate, ethers, polyethers, and any combination of any of the foregoing.

Preferred solvents are water, glycols, esters, alcohols, and mixtures thereof.

For example, the solvent may comprise water and one or more glycol and/or one or more alcohol, such as glycerin, phenoxyethanol, benzyl alcohol, or ethanol. According to one embodiment the solvent system comprises (a) water and (b) (i) a glycol (e.g., glycerin) or (ii) an alcohol (e.g., ethanol).

The personal care composition may be incorporated into an aqueous or oil based system or an emulsion. A suitable solvent for an oil based system is phenoxyethanol and/or benzyl alcohol.

The personal care composition can be a liquid or a solid.

Preferred Personal Care Compositions

Preferred personal care compositions include, but are not limited to, the compositions listed below:

| Composition | |
|---|---|
| No. | Components |
| 1 | Sodium erythorbate, δ-gluconolactone, and, optionally, |
| | sodium sulfite |
| 2 | Sodium erythorbate and, optionally, sodium sulfite |
| 3 | Sodium erythorbate, sodium benzoate, and, optionally, |
| | sodium sulfite |
| 4 | Sodium erythorbate, potassium sorbate, and, optionally, |
| | sodium sulfite |
| 5 | Sodium erythorbate, caffeic acid, and, optionally, |
| | sodium sulfite |
| 6 | Caffeic acid and, optionally, sodium sulfite |
| 7 | Caffeic acid, sodium benzoate, and, optionally, |
| | sodium sulfite |
| 8 | Caffeic acid, potassium sorbate, and, optionally, |
| | sodium sulfite |
| 9 | δ-Gluconolactone and, optionally, sodium sulfite |
| 10 | δ-Gluconolactone, sodium benzoate, and, optionally, |
| | sodium sulfite |
| 11 | δ-Gluconolactone, potassium sorbate, and, optionally, |
| | sodium sulfite |
| 12 | δ-Gluconolactone, caffeic acid, and, optionally, |
| | sodium sulfite |

The weight ratio of the active component of the personal care composition to color stabilizer or potentiator is preferably from about 10:1 to about 1:10, more preferably from about 5:1 to about 1:5, and even more preferably from about 4:1 to about 2:3.

Generally, the personal care composition comprises from about 0.01 to about 99.99% (w/w) of antimicrobial agent. The personal care composition comprises about 99.99 to about 0.01% (w/w) color stabilizer or potentiator. Preferred personal care compositions comprise from about 0.05 to about 20% (w/w) of antimicrobial agent and from about 0.05 to about 20% (w/w) of color stabilizer potentiator, and more preferred color stable personal care compositions comprise from about 0.1 to about 10% (w/w) of antimicrobial agent and from about 0.1 to about 10% (w/w) of color stabilizer or potentiator.

According to a preferred embodiment, the personal care composition contains from about 0.001 to about 10%, preferably from about 0.01 to about 1 or 2%, and more preferably from about 0.05 to about 0.5% by weight of each antimicrobial ingredient (e.g., sodium erythorbate, δ-gluconolactone, or caffeic acid or a salt thereof). When the antimicrobial ingredient is sodium erythorbate, δ-gluconolactone, or caffeic acid or a salt thereof, the personal care composition may contain from about 0.001, 0.005, 0.01, 0.02, 0.03, 0.04, 0.05, 0.06, 0.07, 0.08, 0.09, or 0.1% to about 2, 1, 0.5, 0.4, 0.3, 0.25, 0.2, 0.15, 0.1, 0.09, 0.08, 0.07, 0.06, 0.05, 0.04, 0.03, 0.02, or 0.01% by weight of the antimicrobial ingredient, based upon 100% total weight of personal care composition.

Concentrates

To prepare a product containing the personal care composition, a concentrate of the personal care composition is generally first prepared. The personal care composition of the present invention may be prepared by mixing the color stabilizer or potentiator, AMBP component, and optionally, solvents and additives. The mixture may be heated and/or stirred to expedite mixing. The concentrate may include from about 0.01 to about 100% (i.e., at least about 0.01%) by weight of the active components of the personal care composition, based upon 100% total weight of concentrate. According to one embodiment, the concentrate contains from about 5 to about 80% by weight of the active components of the personal care composition. According to another embodiment, the concentrate includes from about 0.01 to about 5% and more preferably from about 0.05 to about 2% by weight of the active components of the personal care composition. The concentrate may also be preloaded with a biodegradation prevention agent as discussed herein.

Use Dilutions

Before use, the concentrate is diluted, preferably with the same solvent as was used in the concentrate, and/or incorporated into a product. Use dilutions of the composition typically comprise an antimicrobial, preservative, fungicidally, or bactericidally effective amount of the color stable personal care composition.

Generally, the product contains an antimicrobial, preservative, bactericidal, and/or fungicidal effective amount of the personal care composition. In the case of the color stable personal care composition, the product preferably contains a color stable, antimicrobial, preservative, bactericidal, and/or fungicidal effective amount of the color stable personal care composition. Use dilutions generally contain from about 0.0001% or 0.01% to about 2% by weight of the concentrate. According to one preferred embodiment, use dilutions contain from about 0.1 to about 1% by weight of the concentrate. According to another embodiment, the use dilution contains 0.2, 0.25 or 0.30% by weight of the concentrate.

The use dilution generally contains from about 0.01, to about 2.0% by weight of each antimicrobial ingredient, based upon 100% total weight of use dilution.

Uses

The personal care compositions of the present invention are useful in preventing biodegradation, and as fungicidal and bactericidal agents (such as against allergens, tree and plant fungi, and plant and tree bacteria) in the papermaking, textile, agricultural, and coating industries, as well as in personal care, household, industrial, and institutional products. The personal care composition may be incorporated into substrates susceptible to microbial growth to preserve them while maintaining color stability. For example, the personal care composition may be incorporated into or be a personal care product, such as a shampoo, conditioner, cream, lotion (such as body lotion), cosmetic, or soap; a household product, such as a fabric softener, laundry detergent, or hard surface cleaner; or an industrial product, such as paint, coatings, wood, textile, adhesive, sealant, leather, rope, paper, pulp, paper board, sheet rock, ceiling tiles, plastic, fuel, petroleum, oil, rubber working fluid, metal working fluid, starches (such as pet food starch), or mineral slurry, such as a slurry of clay, calcium carbonate, or titanium dioxide (TiO₂).

Moisturizing Compositions

Preferred moisturizing agents for the moisturizing composition of the present invention include, but are not limited to, sodium erythorbate, 6-gluconolactone, and mixtures thereof.

The moisturizing composition preferably contains at least about 0.01% by weight of the moisturizing agent, based on 100% weight of the moisturizing composition. According to a preferred embodiment, the moisturizing composition contains from about 0.001 to about 10%, preferably from about 0.01 to about 1%, and more preferably from about 0.05 to about 0.5% by weight of each moisturizing ingredient (e.g., sodium erythorbate or α-gluconolactone). The amount of the moisturizing agent can be determined by one skilled in the art.

According to one embodiment, the composition contains from about 0.001, 0.005, 0.01, 0.02, 0.03, 0.04, 0.05, 0.06, 0.07, 0.08, 0.09, or 0.1% to about 1, 0.5, 0.4, 0.3, 0.25, 0.2, 0.15, 0.1, 0.09, 0.08, 0.07, 0.06, 0.05, 0.04, 0.03, 0.02, or 0.01% by weight of erythorbic acid or salt thereof (e.g., sodium erythorbate) or δ-gluconolactone based upon 100% total weight of composition.

The moisturizing composition can include any of the additives mentioned above with regard to the personal care composition, such as in the amounts recited above.

The moisturizing composition may be applied to the skin by any method known in the art including, but not limited to, brushing, dipping, and soaking.

The moisturizing compositions of the present invention are useful as moisturizing agents in personal care products. For example, the preservative system may be incorporated into or be a personal care product, such as a shampoo, conditioner, cream, lotion (such as body lotion), cosmetic, or soap; or a household product, such as a fabric softener, laundry detergent, or hard surface cleaner.

Preloaded Concentrates

The present invention also relates to concentrates of active ingredients preloaded with a sufficient amount of a biodegradation prevention agent so that when it is diluted for use, the final formulation includes a biodegradation prevention effective amount of the biodegradation prevention agent.

One embodiment is a preloaded concentrate comprising:
(a) at least about 1, 2, or 5% by weight of a active ingredients other than a bio-degradation prevention agent; and
(b) at least about 1, 2 or 5% by weight of a biodegradation prevention agent.

The additive may be any of the aforementioned additives. According to one embodiment, the additive is not a solvent. Suitable active ingredients and additives include, but are not limited to, a chelating agent, surfactant (including cationic, anionic, nonionic, and amphoteric surfactants), antioxidant, L-carnitine or a salt thereof, an acyl L-carnitine or a salt thereof, hydroxy acids and salts thereof (such as glycolic acid and salts thereof), salicylic acid and salts thereof, proteolytic enzymes, vegetable oils (including, but not limited to, vegetable oil microspheres (oleosomes)), skin lightening agents, and mixtures thereof. Suitable salts of glycolic and salicylic acid include, but are not limited to, sodium and potassium salts.

According to one preferred embodiment, the preloaded concentrate comprises
(i) (a) at least about 1% by weight of arbutin, and
   (b) (1) at least about 1% by weight of glycolic acid or a salt thereof,
      (2) at least about 1% by weight of salicylic acid or a salt thereof,
      (3) at least about 1% by weight of L-carnitine, an acyl L-carnitine, or a mixture thereof,
      (4) at least about 1% by weight of vegetable oil microspheres (oleosomes), or
      (5) a mixture thereof; and
(ii) at least about 1 or 2% by weight of a biodegradation prevention agent.

Preferred mixtures in component (i) include, but are not limited to, (1) arbutin and glycolic acid or a salt thereof, (2) arbutin and salicylic acid or a salt thereof, and (3) arbutin and L-carnitine. For example, the preloaded concentrate can include
(i) (a) from about 1, 2, or 5% to about 10, 15, or 20% by weight of arbutin, and
   (b) (1) from about 1, 2, or 5% to about 10, 15, 20, 25, or 28% by weight of glycolic acid or a salt thereof,
      (2) from about 1, 2, or 5% to about 10, 15, or 20% by weight of salicylic acid or a salt thereof,
      (3) at least about 1, 2, 5, or 10% to about 15, 20, 25, 30, 40, or 50% by weight of L-carnitine, an acyl L-carnitine, or a mixture thereof,
      (4) at least about 1% by weight of vegetable oil microspheres (oleosomes), or
      (5) a mixture thereof; and
(ii) at least about 1 or 2% by weight of a biodegradation prevention agent.

According to another preferred embodiment, the concentrate includes at least about 5, 8, 10, 12, 14, 18, or 20% by weight of L-carnitine, an acyl L-carnitine, a salt thereof, or a mixture thereof. The concentrate can further include a hydroxy acid or a salt thereof, such as glycolic acid or a salt thereof. For example, the concentrate can include at least about 5, 8, 10, 12, 14, 18, or 20% by weight of a hydroxy acid or salt thereof (e.g., glycolic acid or a salt thereof).

Suitable biodegradation prevention agents include, but are not limited to, erythorbic acid and salts thereof, β-gluconolactone, salicylic acid and salts thereof, benzoic acid and salts thereof, dehydroacetic acid and salts thereof, sorbic acid and salts thereof, a formaldehyde donor (such as hydantoins, dimethylhydantoin, and halogenated hydantoins), quaternary ammonium compounds (such as quaternary ammonium chlorides (e.g., benzalkonium chloride and benzethonium chloride) and quaternary ammonium carbonates), iodine containing compounds (such as 3-iodo-2-propynyl butyl carbamate (IPBC) and IPBC/cyclodextrin (available as Glycacil® 2000 from Lonza, Inc. of Fair Lawn, N.J.), isothiazolinones; parabens (such as methylparaben, ethylparaben, propylparaben), isocil, chloroxylenol, chlorhexidine, alcohols (such as phenoxyethanol, benzyl alcohol, phenethyl alcohol, and phenols), chlorobutanol, triclosan, triclocarban, natural oils, natural preservatives (such as cinnamaldehyde), biguanides (such as poly(hexamethylenebiguanide) hydrochloride (PHMB)), sodium hydroxymethylglycinate, and any combination of any of the foregoing.

Generally, the preloaded concentrate is physically and chemically stable. Preferably, the preloaded concentrate is color stable.

The concentrate may also include one or more color stabilizers. Suitable color stabilizers include, but are not limited to, those discussed above, such as sodium sulfite, sodium thioglycolate, calcium gluconate, and sodium gluconate, as well as those described in U.S. Pat. No. 6,017,955.

Preferred color stable preloaded concentrates for the preparation of a color stable personal care composition comprise:
(a) at least 2% by weight of an active ingredient other than a biodegradation prevention agent;
(b) at least 2% by weight of a biodegradation prevention agent selected from erythorbic acid and salts thereof, 6-gluconolactone, salicylic acid and salts thereof, benzoic acid and salts thereof, dehydroacetic acid and salts thereof, and sorbic acid and salts thereof; and
(c) a color stabilizing effective amount of a color stabilizer selected from a sulfite, a sulfate, a thioglycolate, a gluconate, and mixtures thereof.

The concentration of color stabilizer preferably ranges from 1% to 40% by weight, for example from about 1 or 2% to about 10, 20, 30, or 40% by weight, based on 100% total weight of concentrate.

Yet another embodiment is a method of preparing a personal care product comprising the step of mixing:
(a) a preloaded concentrate comprising:
   (i) at least about 1, 2, or 5% by weight and preferably at least 2% by weight of one or more active ingredients other than a biodegradation prevention agent, and
   (ii) at least about 1, 2, or 5% by weight and preferably at least 2% by weight of a biodegradation prevention agent selected from erythorbic acid and salts thereof, δ-gluconolactone, salicylic acid and salts thereof, benzoic acid and salts thereof, dehydroacetic acid and salts thereof, and sorbic acid and salts thereof; and
   (iii) optionally, a color stabilizing effective amount of a color stabilizer selected from a sulfite, a sulfate, a thioglycolate, a gluconate, and mixtures thereof, with
(b) one or more additives.

The resulting personal care product comprises a biodegradation prevention effective amount of the biodegradation prevention agent. The preloaded concentrate may include a color stabilizer as discussed above.

Preferably the resulting personal care product is a shampoo, conditioner, cream, lotion, cosmetic, or soap.

According to one preferred embodiment, at least one of the additives is not a solvent.

The additive is preferably selected from a chelating agent, surfactant, antioxidant, L-carnitine and salts thereof, an acyl L-carnitine and salts thereof, hydroxy acids and salts thereof, proteolytic enzymes, skin lightening agents, and mixtures thereof.

The biodegradation prevention agent may further comprise a preservative selected from formaldehyde donors (such as hydantoins, dimethylhydantoin, and halogenated hydantoins), quaternary ammonium compounds (such as quaternary ammonium chlorides, e.g., benzalkonium chloride and benzethonium chloride, or quaternary ammonium carbonates), iodine containing compounds (such as 3-iodo-2-propynyl butyl carbamate (IPBC) and IPBC/cyclodextrin, isothiazolinones; parabens (such as methylparaben, ethylparaben, propylparaben), isocil, chloroxylenol, chlorhexidine, alcohols (such as phenoxyethanol, benzyl alcohol, phenethyl alcohol and chlorobutanol), phenols, triclosan, triclocarban, natural oils, natural preservatives (such as cinnamaldehyde), biguanides (such as poly(hexamethylenebiguanide) hydrochloride (PHMB)), sodium hydroxymethylglycinate, and any combination of any of the foregoing.

According to one preferred embodiment, the preloaded concentrate comprises (i) at least about 5% by weight of L-carnitine, an acyl L-carnitine, or a mixture thereof, and (ii) at least about 5% by weight of a biodegradation prevention agent. The preloaded concentrate preferably includes a color stabilizer. The concentration of color stabilizer generally ranges from about 1 or 2% to about 10, 20, 30, or 40% by weight, based on 100% total weight of concentrate.

### EXAMPLES

The following examples illustrate the invention without limitation. All parts and percentages are given by weight unless otherwise indicated.

### Example 1

The color stability of sodium erythorbate containing anionic shampoos was tested as follows.

The anionic protein shampoo composition was comprised of 35% by weight of sodium lauryl ether sulfate, 25% by weight of triethanolamine laurylsulfate, 3% by weight coconut diethanolamide (cocamide DEA), 1% by weight of hydrolyzed collagen (available as Polypro 5000™ from Hormel Foods of Austin, Minn.), and 36% by weight of deionized water. Sodium erythorbate was added to the anionic protein shampoo formulation to a concentration of 0.2% w/w.

Each anionic protein shampoo sample was additionally mixed with appropriate amounts of sodium sulfite to achieve the desired concentrations, e.g., 0.3% (Sample 1), 0.2% (Sample 2), 0.1% (Sample 3), 0.05% (Sample 4) sodium sulfite (w/w). One shampoo sample contained no sodium sulfite (Sample 5). The samples were incubated for 9 days at 37° C. Gardner Color tests were performed on the samples following incubation.

The results are shown in Table 1 below.

TABLE 1

| Sample | | Gardner Color | |
|---|---|---|---|
| No. | Color Stabilizer | Results | Comments |
| 1 | 0.3% Sodium Sulfite | 1-2 | No change |
| 2 | 0.2% Sodium Sulfite | 1-2 | No change |
| 3 | 0.1% Sodium Sulfite | 1-2 | No change |
| 4 | 0.05% Sodium Sulfite | 2-3 | No change |
| 5 | None | 2-3 | became darker at room |
| | | | temperature within |
| | | | 2 days |

### Example 2

Glycerol monostearate (GMS) cream moisturizing compositions containing 2% glycerin (Sample 1), 1% sodium erythorbate (Sample 2), 1% δ-gluconolactone (Sample 3), or 2% of a biodegradation prevention mixture containing 75% (by weight) sodium benzoate and 25% (by weight) sodium erythorbate were tested as follows.

The GMS cream was prepared as follows. Polyoxyethylene glyceryl monostearate, glyceryl monostearate, cetearyl alcohol, and myristyl propionate were mixed and heated to 60° C. in a first container. In four separate containers, glycerin, sodium erythorbate, δ-gluconolactone, or the aforementioned biodegradation prevention mixture were mixed with sterile deionized water and heated to 60° C. The solution in the first container was poured into each container containing glycerin, sodium erythorbate, δ-gluconolactone, or the biodegradation prevention mixture to produce the desired concentrations, e.g., 2% glycerin (w/w) (Sample 1), 1% sodium erythorbate (w/w) (Sample 2), 1% δ-gluconolactone (w/w) (Sample 3), or 2% biodegradation prevention mixture (w/w) (Sample 4). The containers were maintained at 60° C. for 10 minutes, and then allowed to cool. The pH of the solution was adjusted to pH 7 with sodium hydroxide to yield the GMS cream.

The moisturizing compositions were applied once to skin and skin moisture measurements were taken over a 7 day period following administration. Skin moisture measurements were performed with a Corneometer.

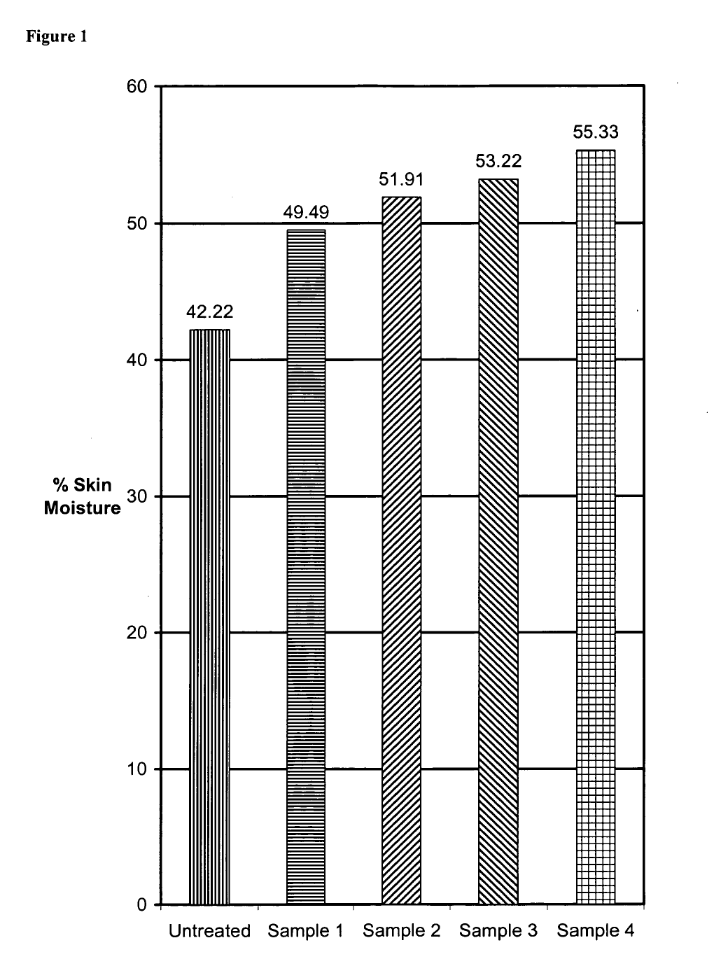

The results are shown in FIG. 1.

### Example 3

The color stability of caffeic acid containing shampoos was tested as follows.

The anionic protein shampoo composition was comprised of 35% by weight of sodium lauryl ether sulfate, 25% by weight of triethanolamine laurylsulfate, 3% by weight coconut diethanolamide (cocamide DEA), 1% by weight of hydrolyzed collagen (available as Polypro 5000™ from Hormel Foods of Austin, Minn.), and 36% by weight of deionized water. Caffeic acid was added to the anionic protein shampoo formulation to a concentration 0.2% caffeic acid by weight. Samples containing varying amounts of caffeic acid and sodium sulfite as shown in Table 2 below were prepared by adding the appropriate amounts of these ingredients to the anionic protein shampoo.

The initial color of each sample was noted, and each sample was incubated for 10 weeks at room temperature. At the end of the incubation, the color of each sample was noted, and compared to its initial color.

The results are shown in Table 2 below.

TABLE 2

| | | | End Color |
|---|---|---|---|
| Sample | Specific Example | Beginning Color | (after 10 weeks) |
| 1 | Shampoo | Very pale yellow/almost | Same as |
| | | water white | beginning |
| 2 | Shampoo w/0.2% | Very pale yellow/almost | Dark amber |
| | Caffeic Acid | water white | |
| 3 | Shampoo w/0.2% | Slight pink tint/almost | Same as |
| | Caffeic Acid and | water white | beginning |
| | 0.2% Sodium | | |
| | Sulfite | | |

### Example 4

The antimicrobial and antifungal properties of creams containing LAG (5% arbutin, 28% glycolic acid, pH 4) or LAGnp (5% arbutin, 28% glycolic acid, 5% biodegradation prevention mixture (60% (by weight) δ-gluconolactone, 20% (by weight) sodium erythorbate, and 20% (by weight) sodium sulfite), at pH 4) were tested as follows.

GMS creams were formulated with varying concentrations of LAG or LAGnp according to the procedure described in Example 2. Samples were formulated containing 20% (w/w) LAGnp (Sample C), 17% (w/w) LAGnp (Sample D), 15% LAGnp (Sample E), and 10% LAGnp (Sample F). Control samples containing no additives (Sample A) or 20% LAG (Sample B) were also formulated. Samples were tested against bacteria or fungi as described below. The number of bacterial or fungal cells was determined after 7 days.

Bacterial Testing

Each cream sample was tested as follows. A standardized mixed bacterial solution was prepared according to the following procedure. 3 agar stabs of S. aureus (ATCC # 6538), P. aeruginosa (ATCC # 9027), and E. coli (ATCC # 8739) were separately incubated at about 35° C. for about 24 hours. Each stab was then washed with 3 mL of sterile 0.85% saline solution. The washes of the 3 stabs were pooled together to form an organism mixture. The absorbance of the organism mixture at 530 nm was adjusted to about 1.00 by adding saline. The spectrometer was calibrated with a saline blank. A 5 mL aliquot of the organism mixture was mixed together to produce the standardized mixed bacterial solution. Then, 40 g of each sample was inoculated with 0.2 mL of the standardized mixed bacterial solution and mixed. 1 g of the mixture was added to a sterile 20×150 mm screw cap test tube. 9 mL of sterile D/E neutralizer broth was added to the test tube and mixed to form a 10⁻¹ dilution. Serial dilutions were prepared through to a 10⁻⁶ dilution with phosphate buffered water. The serial dilutions were plated onto Tryptic Soy Agar and incubated for 2 days at about 35° C. Bacteria counts were performed after 0 and 7 days.

Fungal Testing

Each sample was tested as follows. A standard mixed fungal solution was prepared according to the following procedure. 2 agar slants of Candida albicans and 4 agar slants of Aspergillus niger were separately incubated at about 25° C. for about 48 hours and 7 days, respectively. Each slant was washed with 3 mL of sterile 0.85% saline solution, collected and macerated in a tissue grinder. Sufficient amounts of 0.85% saline solution were added to each slant to obtain a visual count under a microscope with a Neubauer Hemocytometer of each innoculum of C. albicans and A. niger. Equal volumes of each standardized inoculum of C. albicans and A. niger were mixed together to form the standardized mixed fungal solution.

40 g of each sample was inoculated with 0.4 mL of the standardized mixed fungal solution and mixed. 1 g of the mixture was added to a sterile 20×150 mm screw cap test tube.

9 mL of sterile D/E neutralizer broth was added to the test tube and mixed to form a 10⁻¹ dilution. Serial dilutions were prepared through to a 10⁻⁶ dilution with phosphate buffered water. The serial dilutions were plated onto Sabourand dextrose agar and incubated 5 days at about 25° C. Fungal counts were performed after 0 and 7 days.

The results are shown in Table 3 and 4 below.

TABLE 3
Bacteria Results

Colony Forming
Units/g

| Sample | Formulation | Day 0 | Day 7 |
|---|---|---|---|
| A | Unpreserved cream | >3 × 10⁶ | <10 |
| B | Cream with 20% LAG | + | <10 |
| C | Cream with 20% LAGnp | + | <10 |
| D | Cream with 17% LAGnp | + | <10 |
| E | Cream with 15% LAGnp | + | <10 |
| F | Cream with 10% LAGnp | >3 × 10⁶ | <10 |

TABLE 4
Fungi Results

Colony Forming
Units/g

| Sample | Formulation | Day 0 | Day 7 |
|---|---|---|---|
| A | Unpreserved cream | 1.6 × 10⁵ | 3.7 × 10⁴ |
| B | Cream with 20% LAG | + | 20 |
| C | Cream with 20% LAGnp | + | <10 |
| D | Cream with 17% LAGnp | + | <10 |
| E | Cream with 15% LAGnp | + | <10 |
| F | Cream with 10% LAGnp | 2.4 × 10⁵ | <10 |

### Example 5

The color stabilizing properties of creams containing LAG (5% arbutin, 28% glycolic acid, pH 4) and a biodegradation prevention mixture (BPM) (60% (by weight) δ-gluconolactone, 20% sodium erythorbate, and 20% sodium sulfite) were tested as follows.

Samples were formulated containing the 95 or 90% (w/w) LAG with 5% or 10% (w/w) of the biodegradation prevention mixture. The samples were stored for one month at room temperature, 37° C. or 5° C. The color of the samples was evaluated at day 0, 1 week, 2 weeks, 3 weeks, and 1 month.

The results are shown in Table 5 below.

TABLE 5

| Sample | Temperature | Contents | Day 0 | 1 Week | 2 Weeks | 3 Weeks | 1 Month |
|---|---|---|---|---|---|---|---|
| 1 | Room | LAG | Water | Water | Light | Light | Peach |
| | | | white | white | peach | peach | |
| 2 | Room | LAG with | Water | Water | Water | Water | Water |
| | | 5% BPM | white | white | white | white | white |
| 3 | Room | LAG with | Water | Water | Water | Water | Water |
| | | 10% BPM | white | white | white | white | white |
| 4 | 37° C. | LAG with | Water | Water | Water | Water | Water |
| | | 5% BPM | white | white | white | white | white |
| 5 | 37° C. | LAG with | Water | Water | Water | Water | Water |
| | | 10% BPM | white | white | white | white | white |
| 6 | 5° C. | LAG with | Water | Water | Water | Water | Water |
| | | 5% BPM | white | white | white | white | white |
| 7 | 5° C. | LAG with | Water | Water | Water | Water | Water |
| | | 10% BPM | white | white | white | white | white |

1. A color stable personal care composition comprising:
(a) an anti-oxidation, moisturizing, and/or biodegradation preventing (AMBP) component comprising:
(1) erythorbic acid or a salt thereof;
(2) caffeic acid or a salt thereof;
(3) δ-gluconolactone; or
(4) a mixture of at least two of the foregoing; and
(b) a color stabilizer selected from a sulfite, a sulfate, a thioglycolate, a gluconate, or mixture thereof.

2. The color stable personal care composition of claim 1, wherein the AMBP component comprises sodium erythorbate.

3. The color stable personal care composition of claim 1, wherein the AMBP component further comprises benzoic acid or a salt thereof, or sorbic acid or a salt thereof.

4. The color stable personal care composition of claim 1 wherein the color stable personal care composition further comprises one or more additional antimicrobial agents.

5. The color stable personal care composition of claim 4, wherein the additional antimicrobial agent is selected from 3-iodo-2-propynyl butyl carbamate, dehydroacetic acid and salts thereof, salicylic acid and salts thereof, parabens, polyaminopropyl biguanide, quaternary ammonium compounds, hydantoins, isothiazolinones, antimicrobial alcohols, natural oils, and mixtures thereof.

6. The color stable personal care composition of claim 1, wherein the color stabilizer is sodium sulfite, sodium thioglycolate, calcium gluconate, sodium gluconate, or a mixture of at least two of the foregoing.

7. The color stable personal care composition of claim 1, further comprising a solvent.

8. The color stable personal care composition of claim 7, wherein the solvent is selected from water, glycols, esters, alcohols, and mixtures thereof.

9. A non-therapeutic method of moisturizing skin comprising applying a moisturizing composition comprising a moisturizing effective amount of erythorbic acid or salt thereof, δ-gluconolactone, or a mixture thereof.

10. A personal care product comprising a moisturizing composition comprising from 0.01% to 1% by weight of sodium erythorbate, δ-gluconolactone, or a mixture thereof.

11. A color stable preloaded concentrate for the preparation of a color stable personal care composition, said concentrate comprising:
(a) at least 2% by weight of an active ingredient other than a biodegradation prevention agent;
(b) at least 2% by weight of a biodegradation prevention agent selected from erythorbic acid and salts thereof, δ-gluconolactone, salicylic acid and salts thereof, benzoic acid and salts thereof, dehydroacetic acid and salts thereof, and sorbic acid and salts thereof; and
(c) a color stabilizing effective amount of a color stabilizer selected from a sulfite, a sulfate, a thioglycolate, a gluconate, and mixtures thereof.

12. The color stable preloaded concentrate of claim 11, wherein the color stabilizer is sodium sulfite, sodium thioglycolate, calcium gluconate, sodium gluconate, or a mixture of at least two of the foregoing.

13. The color stable preloaded concentrate of claim 11, wherein the concentration of color stabilizer ranges from 1 to 40% by weight, based on 100% total weight of concentrate.

14. A method of preparing a personal care product comprising the step of mixing:
(a) a preloaded concentrate comprising:
(i) at least 2% by weight of one or more active ingredients other than a biodegradation prevention agent, and
(ii) at least 2% by weight of a biodegradation prevention agent selected from erythorbic acid and salts thereof, δ-gluconolactone, salicylic acid and salts thereof, benzoic acid and salts thereof, dehydroacetic acid and salts thereof, and sorbic acid and salts thereof; and
(iii) optionally, a color stabilizing effective amount of a color stabilizer selected from a sulfite, a sulfate, a thioglycolate, a gluconate, and mixtures thereof, with
(b) one or more additives,
wherein the personal care product comprises a biodegradation prevention effective amount of the biodegradation prevention agent.

15. The method of claim 14, wherein the personal care product is a shampoo, conditioner, cream, lotion, cosmetic, or soap.

16. The method of any of claim 14, wherein at least one of the additives is not a solvent.

17. The method of claim 14, wherein the additive is selected from a chelating agent, surfactant, antioxidant, L-carnitine and salts thereof, an acyl L-carnitine and salts thereof, hydroxy acids and salts thereof, proteolytic enzymes, skin lightening agents, and mixtures thereof.

18. The method of claim 14, wherein the biodegradation prevention agent further comprises a preservative selected from formaldehyde donors, quaternary ammonium compounds, iodine containing compounds, isocil, chloroxylenol, chlorhexidine, alcohols, phenols, triclosan, triclocarban, natural oils, natural preservatives, biguanides, sodium hydroxymethylglycinate, and any combination of any of the foregoing.

19. The color stable personal care composition of claim 1, wherein the AMBP component further comprises sodium benzoate.

20. The color stable personal care composition of claim 1, wherein the AMBP component further comprises potassium sorbate.
